(19) United States
(12) Patent Application Publication
(10) Pub. No.: US 2005/0224594 A1
(43) Pub. Date: Oct. 13, 2005

(54) Air freshener removably coupleable with a paper towel roll

(76) Inventor: Thomas H. Wolf, Highland Park, IL (US)

Correspondence Address: James B. Conte;BARNES & THORNBURG LLP, P.O. Box 2786, Chicago, IL, 60690-2786, US

(21) Appl. No.: 10/821,093

(22) Filed: Apr. 7, 2004

(51) Int. Cl. A61L 9/04; A24F 25/00

(52) U.S. Cl. 239/34; 239/47; 239/55

(57) ABSTRACT

An air freshener has a base. The base has scented material therein. An abutment is connected to said base. A guide which accommodates a portion of a paper towel holder is connected to said abutment. The air freshener in an installed has an external surface of the abutment in frictional contact with an internal surface of the paper towel tube so that the air freshener will rotate when the paper towel tube is rotated by conventional means.

### FIELD OF INVENTION

The present invention concerns an air freshener having an abutment portion removably coupleable within an end portion of a tube in a paper towel roll.

### BACKGROUND

There are many products on the market aimed at improving the scent of the air in household rooms. One such product is a scented toilet paper roller. A known roller has a cavity containing scented beads and a number of openings to allow the scent from the beads to be released into the air. The roller supports a roll of toilet paper in a conventional toilet paper holder. An example of a known scented roller is described in U.S. Pat. No. 4,759,510, Singer. The patent is directed towards a universal scent-emitting toilet paper roller. The roller is described as including three components molded of thermoplastic material. Two of these components are identical vented hollow closed ended generally cylindrical roller halves, each half containing tongues and slots which permit the two roller halves to be axially slidably joined together. The third thermoplastic component is a helical coiled spring designed to fit inside the joined together roller halves and urge the two halves into their maximum axially extended position. A recessed truncated conical projection axially centered on the closed end of each roller half fits the assembled roller into a rolled paper dispenser. A quantity of scent-emitting pellets are contained within the hollow interior of the joined together roller halves to be tumbled about as the roller is rotated by the removal of paper, thus causing scent to be emitted into the atmosphere through vents in the roller.

### SUMMARY OF INVENTION

One embodiment of the present invention is an air freshener. The air freshener has a base. Extending from the base is an abutment. A guide which accommodates a portion of a paper towel holder is connected to said abutment. The base forms a chamber. Within the chamber is scented material. Vents open into the chamber.

The air freshener has an installed position wherein in the installed position the abutment extends into an end portion of a conventional tube of a paper towel roll. The abutment has an external surface which contacts the internal surface of the paper towel tube to form a frictional fit therewith such that the abutment will rotate when the roll of paper towel roll is rotated during conventional use.

### BRIEF DESCRIPTION OF THE DRAWINGS

FIG. 1 is a side perspective view of the air freshener aligned with an insert bracket of a wall mounted paper towel holder and the paper towel roll, wherein the paper towel roll has been partially cut away.

FIG. 2 is a front plan view looking into the aligned air freshener shown in FIG. 1.

FIG. 3 is a rear plan view of the aligned air freshener shown in FIG. 1.

FIG. 4 is a cross-sectional view of the air freshener shown in FIG. 2 without the paper towel bracket.

FIG. 5 is a side perspective view of the air freshener wherein one of the two side walls forming the base has been removed from the base.

FIG. 6 is a front side view of the air freshener showing a fill plug removed from an aperture.

FIG. 7 is a front side perspective view of the air freshener in a metalized wrapping wherein a portion of the wrapping has been cut out.

FIG. 8 is one of the scented beads depicted in FIG. 4.

FIG. 9 is a partial sectional view of the scented bead of FIG. 8 showing a layer of scented oil around the plastic core of the scented bead.

FIG. 10 is a perspective view of the air freshener aligned with an upright paper towel holder and paper towel roll.

FIG. 11 is a perspective view of the air freshener aligned around the spindle of a wall mounted paper towel holder having a paper towel roll therein.

FIG. 12 is a front and side perspective view of an alternative embodiment of the air freshener in FIG. 1; the abutment being a pair of resilient arms having internal support brackets extending therefrom.

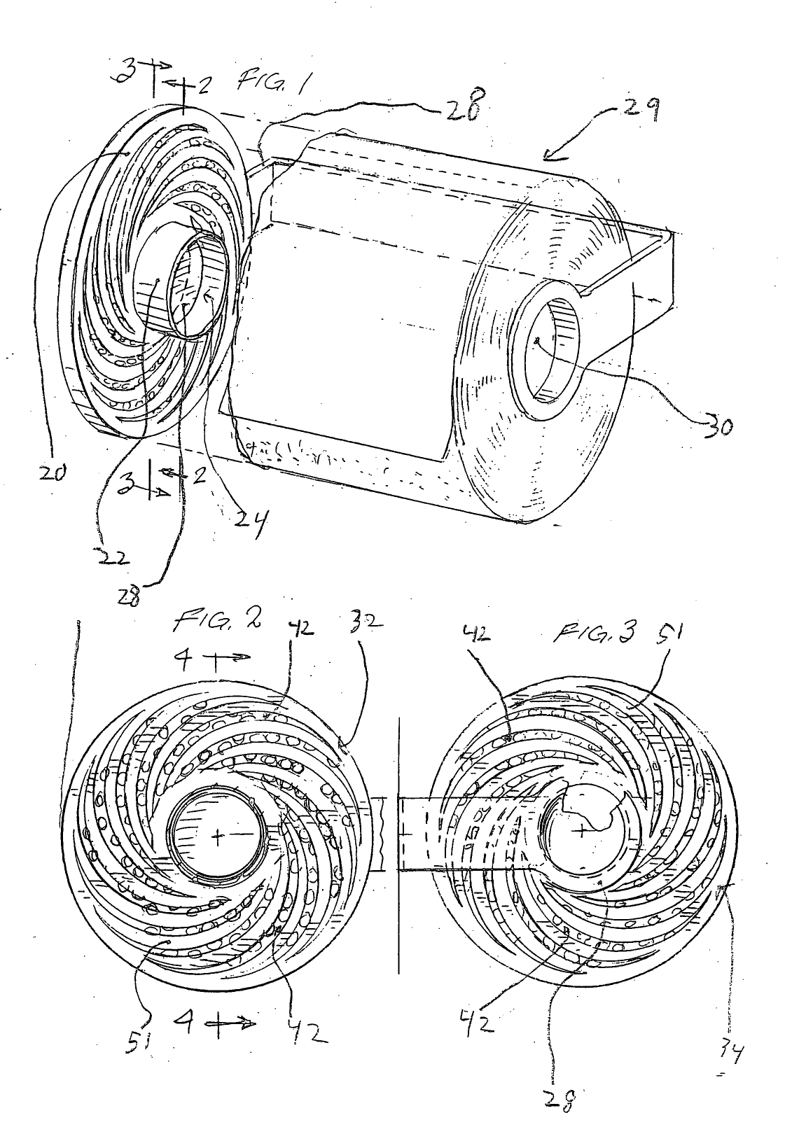

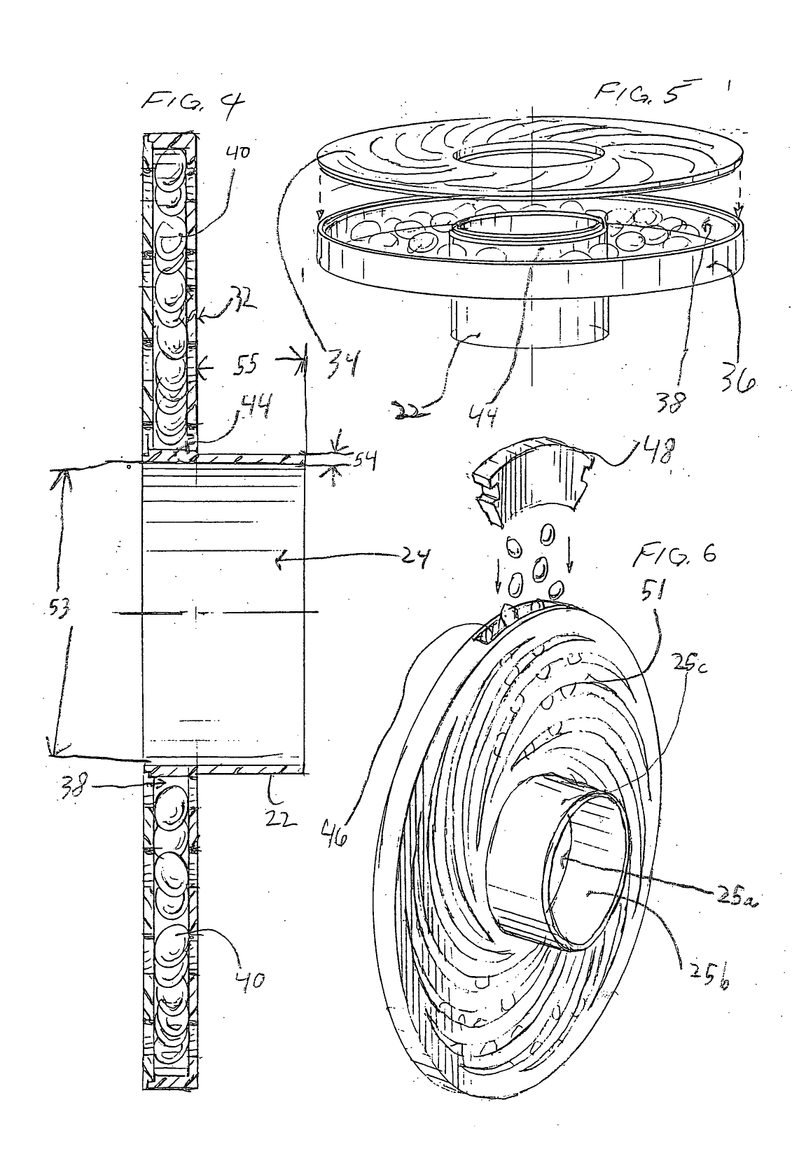

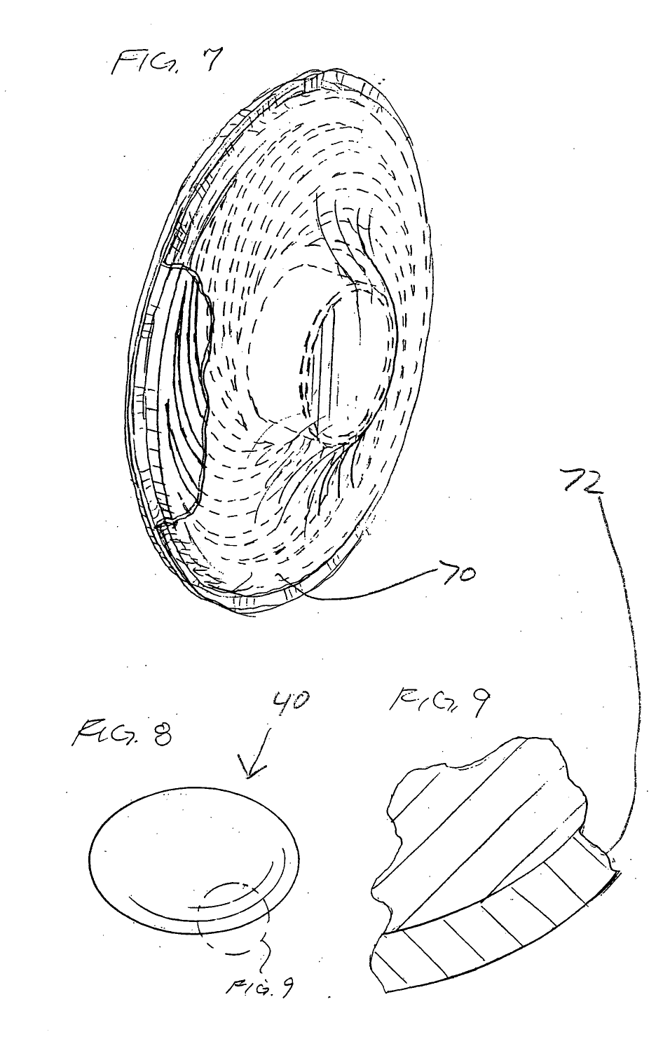

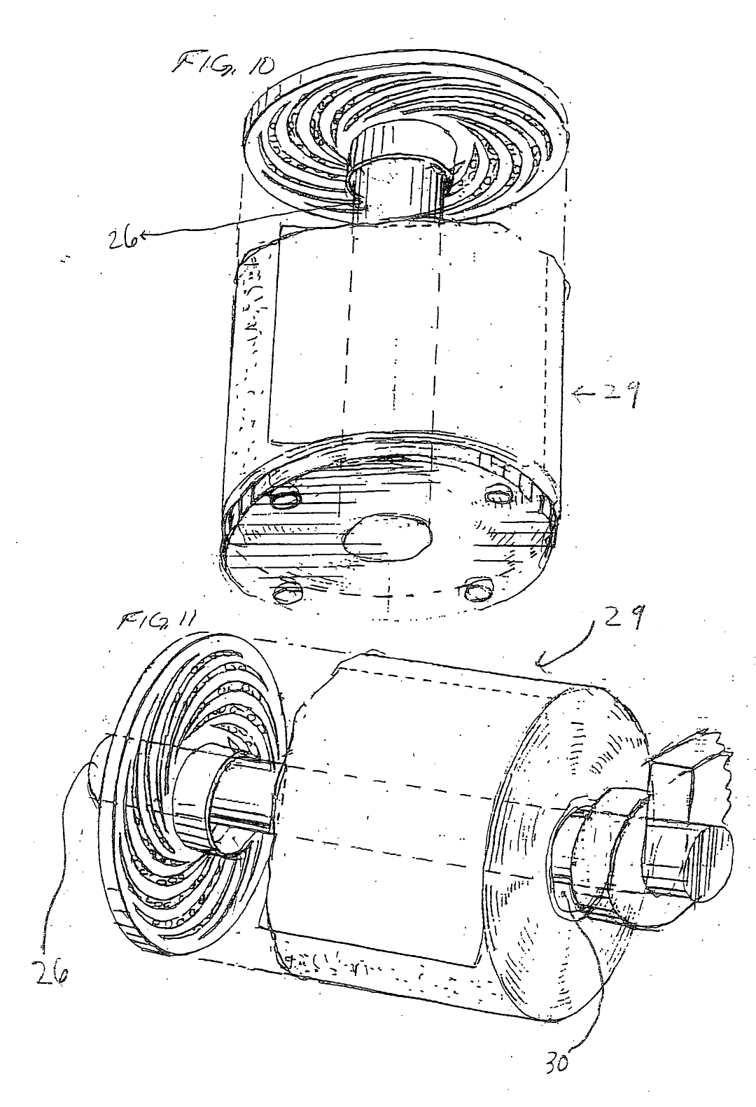

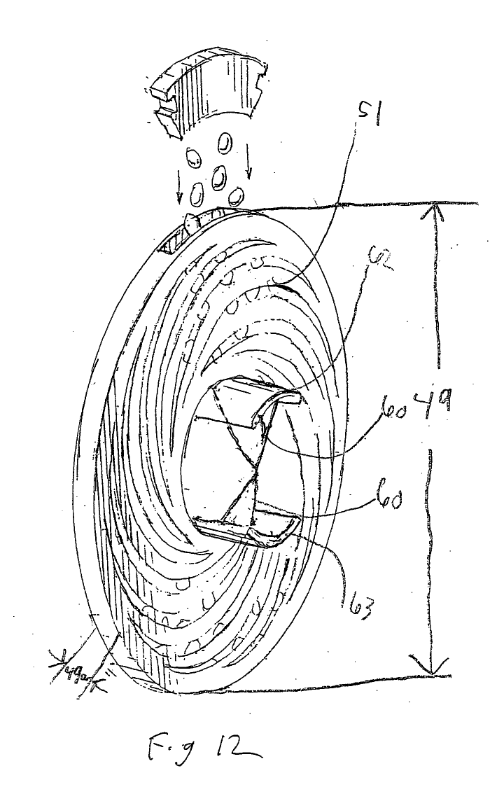

### DETAILED DESCRIPTION OF THE EMBODIMENT

One example of the invention is further described in detail as follows.

The air freshener has a base 20, an abutment 22 and a guide 24. The guide 24 allows the air freshener to be oriented around a portion of a paper towel holder, such as a portion of a spindle 26 or a portion of a towel holder bracket 28. The guide 24 in the present embodiment is in the form of a bore 25a and counter bore 25b through the air freshener. The bore can be referred to as a passage or aperture.

In an installed position at least a portion of the abutment 22 is disposed within an end portion (cut away in the drawings) of the tube 30 in the paper towel roll 29. The abutment frictionally couples with the tube such that the air freshener will rotate with the tube when the tube is rotated around spindle 26 or insert bracket 28 of a conventional paper towel holder. The base 20 is external to the paper towel roll at the one end (cut away in the drawings) of the paper towel roll.

The base 20 of the described embodiment has a first end wall 32 and a second end wall 34. A side wall 36 joins the first end wall and second wall. The first end wall 32, second end wall 34 and side wall 36 enclose a chamber 38. The chamber 38 has disposed therein scented material 40. Within the chamber is an internal barrier 44 which prevents the scented material 40 from falling into the aperture 25a. The internal barrier 44 is formed from an end of the tube forming the abutment 22. The abutment portion 22 of the tube extends from one side of the first end wall. The internal barrier portion 44 of the tube extends from the other side of the first end wall.

Vents 42 in the end walls allow for ambient air to be in fluid communication with the scented material 40.

The base, can have an aperture 46 in the side wall into which a fill plug 48 can be disposed. The aperture and fill plug allow for disposal of the scented material 40 in the base chamber 20.

The base member would have a diameter 49 of about 4½″ and an axial length 49a of between 3/16″ to ¼″. The venting in the base can have a variety of configurations. The shown embodiment shows a swirled-like venting on both the first and second end walls. The venting is formed by way of curvilinear openings 42 in the end walls. The curvilinear openings are separated by curvilinear ribs 51. The air freshener can be made with only venting either the first or second end walls as opposed to both. Additionally, the side wall could be vented. Further, only portions of the base member could be vented.

The abutment 22 extends away from the first and second end wall and is transverse to the first and second end wall. The abutment 22 is tubular. The internal surface 25b of the tubular abutment 22 defines a portion of the guide which can accommodate a conventional paper towel spindle 26 or end bracket 28. The external surface 25c of the tubular abutment provides frictional coupling with the internal surface of the paper towel tube. The frictional coupling should have sufficient opposing forces so the air freshener remains stationary relative to the paper towel tube when the paper towel tube is rotated. It is contemplated that the tubular abutment would have an interior diameter 53 of approximately 1 7/16 of an inch. The thickness 54 of the tubular abutment member would be approximately 1/32 of an inch. The tubular abutment would have an axial length 55 of about ½ of an inch.

Although the guide in one shown embodiment is completely open prior to disposal over the paper towel spindle, it is possible that the guide could be closed and open upon insertion of the spindle or bracket into the guide. For instance, the guide (FIG. 12) could include resilient internal members 60 which open up upon insertion of the spindle through the abutment 62, 63. In the alternative embodiment, the abutment is a pair of juxtapositioned arms 62, 63. The resilient members 60 after insertion would exert an outward force on the abutment to cause or assist the external surface of the abutment to form a frictional fit with the internal surface of the paper towel tube. The resilient members of course could also be positioned or constructed differently.

The air freshener can be sealed with a metalized wrapper 70. The wrapper can be disposed around the air freshener so as to provide a substantially airtight barrier around the freshener. The metalized wrapper is preferably aluminum coated with polyester. The scented material disposed within the base consists of a plurality of scented beads 40. The beads are made out of a resilient soft plastic. The beads are scented. The scented beads have applied thereto a layer of scented oil 72.

The invention disclosed herein has been described with reference to a particular example. It is understood that the invention may be embodied in a variety of other examples not specifically described herein. The scope of the invention, thus is not limited by the example described herein. The invention is intended to only be limited as required by the appended claims.

1. An air freshener comprising:
a base having scented material therein;
an abutment connected to said base; and
guide which accommodates a portion of a paper towel holder, connected to said abutment.

2. The air freshener of claim 1 wherein said air freshener further comprises:
an installed position wherein,
said abutment has an external surface in frictional contact with an internal surface of the paper towel tube, and
wherein said air freshener will rotate when said paper towel tube is rotated by conventional means.

3. The air freshener of claim 1 wherein the base further comprises:
a first end wall;
a second end wall spaced from the first end wall;
a side wall joining the first and second end walls;
a chamber formed by said first end wall, said second end wall and said side wall.

4. The air freshener of claim 1 wherein the abutment has a width sufficient to form a frictional coupling with a conventional paper towel tube when at least a portion of said abutment is disposed in said tube.

5. The air freshener of claim 1 wherein the scented material is a plurality of scented beads.

6. The air freshener of claim 1 wherein the abutment is tubular and an internal surface of the abutment forms at least a portion of said guide.
